(19) United States
(12) Patent Application Publication
(10) Pub. No.: US 2026/0038671 A1
(43) Pub. Date: Feb. 5, 2026

(54) THE METHOD OF AND THE COMPUTING DEVICE FOR GENERATING SURGICAL CONDITION INFORMATION

(71) Applicants: NATIONAL TAIPEI UNIVERSITY OF TECHNOLOGY, Taipei City (TW); TAIPEI MEDICAL UNIVERSITY, Taipei City (TW)

(72) Inventors: CHENG-CHUN CHANG, Taipei City (TW); MENG-HUANG WU, Taipei City (TW); CHAO-WEI CHEN, Taipei City (TW); CHIH-CHENG TAI, Campbell, CA (US)

(73) Assignees: NATIONAL TAIPEI UNIVERSITY OF TECHNOLOGY, Taipei City (TW); TAIPEI MEDICAL UNIVERSITY, Taipei City (TW)

(21) Appl. No.: 19/120,235

(22) PCT Filed: Oct. 12, 2023
(86) PCT No.: PCT/IB2023/000586

§ 371 Date: Apr. 11, 2025

Related U.S. Application Data
(60) Provisional application No. 63/415,598, filed on Oct. 12, 2022.

(51) Int. Cl. G16H 30/40; G06T 7/00; G06V 10/82; G06V 20/40

(52) U.S. Cl. CPC G16H 30/40; G06T 7/0012; G06V 10/82; G06V 20/44; G06T 2207/10048; G06T 2207/10068; G06T 2207/20081; G06T 2207/20084

(57) ABSTRACT

The present application provides a method of generating surgical condition information. A surgical condition information is generated based on a surgical image set that is associated with a surgical site by a computing device. The method includes receiving the surgical image set and automatically generating the surgical condition information based on the surgical image set. The surgical condition information contains a bleeding warning, a bleeding point identification, a blood flow path indication, a prompting bleeding point, a bleeding amount indication, or a bleeding speed indication. In addition, a computing device of generating surgical condition information is also provided in the present application.

### CROSS-REFERENCE TO RELATED APPLICATION

This non-provisional application claims priority under 35 U.S.C. § 119(e) on US provisional Patent Application No(s) 63/415,598 filed on Oct. 12, 2022, the entire contents of which are hereby incorporated by reference.

### BACKGROUND OF THE INVENTION

### 1. Field of the Invention

The present disclosure relates to a method of and a computing device for generating information, and in particular to a method of and a computing device for generating a surgical condition information.

### 2. Description of the Related Art

Conventionally, a user, such as a surgeon, uses endoscopic images to evaluate a patient's medical condition regarding an internal organ of the patient. However, in case of internal bleeding on the part of the patient, it will be difficult for the user to use the endoscopic images to evaluate bleeding status (for example, a bleeding warning, a bleeding point identification, a blood flow path indication, a prompting bleeding point, a bleeding amount indication, or a bleeding speed indication). Therefore, even though the patient's internal bleeding can be confirmed by the endoscopic images, the user is not provided with sufficient information to make a decision on any appropriate procedure for coping with the internal bleeding.

### BRIEF SUMMARY OF THE INVENTION

In view of the aforesaid drawbacks of the prior art, it is imperative to provide methods and computing devices to provide a surgical condition information (for example, a bleeding warning, a bleeding point identification, a blood flow path indication, a prompting bleeding point, a bleeding amount indication, or a bleeding speed indication).

To this end, the present disclosure provides methods of and computing devices for generating a surgical condition information to provide the surgical condition information based on a surgical image set.

In some embodiments, the present disclosure provides a method of generating surgical condition information. A surgical condition information is generated based on a surgical image set that is associated with a surgical site by a computing device. The method includes receiving the surgical image set and automatically generating the surgical condition information based on the surgical image set. The surgical condition information contains a bleeding warning, a bleeding point identification, a blood flow path indication, a prompting bleeding point, a bleeding amount indication, or a bleeding speed indication.

In some embodiments, the step of automatically generating the surgical condition information based on the surgical image set includes inputting the surgical image set into a first image identifying model and outputting the bleeding warning by the first image identifying model. The bleeding warning is automatically generated based on the surgical image set by the first image identifying model. The first image identifying model is a deep learning model that has been trained by plural pieces of first training images.

In some embodiments, the step of automatically generating the surgical condition information based on the surgical image set includes inputting the surgical image set into a second image identifying model and outputting the bleeding point identification by the second image identifying model. The bleeding point identification is automatically generated based on the surgical image set by the second image identifying model. The second image identifying model is a deep learning model that has been trained by plural pieces of second training images.

In some embodiments, the method of generating surgical condition information further includes marking the bleeding point identification that is corresponding to the surgical image set on the surgical image set.

In some embodiments, the step of automatically generating the surgical condition information based on the surgical image set includes inputting a first image and a second image into an optical flow tracking calculating model and outputting the blood flow path indication by the optical flow tracking calculating model. The surgical image set contains the first image and the second image. The blood flow path indication is automatically generated based on the first image and the second image by the optical flow tracking calculating model.

In some embodiments, the method of generating surgical condition information further includes generating the prompting bleeding point based on the blood flow path indication.

In some embodiments, the method of generating surgical condition information further includes marking the blood flow path indication on the surgical image set.

In some embodiments, the step of automatically generating the surgical condition information based on the surgical image set includes inputting the surgical image set into a third image identifying model, outputting a bleeding area indication by the third image identifying model, and converting the bleeding area indication into the bleeding amount indication. The bleeding area indication is automatically generated based on the surgical image set by the third image identifying model. The third image identifying model is a deep learning model that has been trained by plural pieces of third training images.

In some embodiments, the method of generating surgical condition information further includes generating the bleeding speed indication based on the bleeding amount indication that is corresponding to a third image and the bleeding amount indication that is corresponding to a fourth image. The surgical image set contains the third image and the fourth image.

In some embodiments, the method of generating surgical condition information further includes receiving an infrared image set, performing an image processing for the infrared image set, and displaying the surgical condition information on the infrared image set.

Furthermore, the present disclosure also provides a computing device for generating surgical condition information. A surgical condition information is generated based on a surgical image set that is associated with a surgical site by the computing device. The computing device signally connects with an endoscope device via a signal transmitting path. The endoscope device is configured to provide a surgical image set. The computing device includes a processing module, and a storage module. The storage module is configured to signally connect with the processing module. A code is stored in the storage module, and after the processing module executes the code stored in the storage module, the computing device performs the steps as described below: receiving the surgical image set and automatically generating the surgical condition information based on the surgical image set. The surgical condition information contains a bleeding warning, a bleeding point identification, a blood flow path indication, a prompting bleeding point, a bleeding amount indication, or a bleeding speed indication.

In some embodiments, the computing device further performs the steps as described below: receiving an infrared image set, performing an image processing for the infrared image set, and displaying the surgical condition information on the infrared image set.

Furthermore, the present disclosure also provides a non-transitory computer-readable storage medium. After a computing device loads and executes a program that is stored in the non-transitory computer-readable storage medium, the computing device can implement any one of the methods of generating surgical condition information described above.

Furthermore, the present disclosure also provides a computer program product. After a computing device executes the computer program product, the computing device can implement any one of the methods of generating surgical condition information described above.

The present disclosure provides an improvement in the technical field about processing and analyzing the surgical image set, and thus the present disclosure can provide the surgical condition information with a user, such as a surgeon.

In addition, the present disclosure further provides a method of coloring a tissue image. The method includes receiving a first visible light image including a first visible tissue image of a tissue, obtaining first tissue data including first tissue structure information and first tissue color information based on the first visible tissue image, receiving a first low chroma image including a first low chroma tissue image of the tissue, and coloring the first low chroma tissue image on the first low chroma image based on the first tissue structure information and the first tissue color information.

In some embodiments, the method of coloring a tissue image further includes receiving a second visible light image including a second visible tissue image of the tissue and a second blood image, receiving a second low chroma image including a second low chroma tissue image of the tissue, and coloring the second low chroma tissue image on the second low chroma image based on the first tissue structure information, the first tissue color information, the second visible tissue image, and the second blood image.

The present disclosure provides an improvement in the technical field about processing and analyzing the surgical image set, and thus the present disclosure can provide the tissue image hidden under the blood when their image field of view is at least partially hidden under blood.

### BRIEF DESCRIPTION OF THE DRAWINGS

FIG. 1 is a schematic diagram illustrating a system for generating surgical condition information according to an embodiment of the present disclosure.

FIG. 2A is a perspective view of the endoscope device as shown in FIG. 1 according to an embodiment of the present disclosure.

FIG. 2B is a schematic diagram illustrating internal components of the main body as shown in FIG. 2A according to an embodiment of the present disclosure.

FIG. 2C is a schematic diagram illustrating the computing device as shown in FIG. 1 according to an embodiment of the present disclosure.

FIG. 3 is a flowchart illustrating a method of generating surgical condition information according to an embodiment of the present disclosure.

FIG. 4A is a flowchart illustrating the method of generating the bleeding warning according to an embodiment of the present disclosure.

FIG. 4B is a schematic diagram illustrating the surgical image set with the bleeding warning according to an embodiment of the present disclosure.

FIG. 5A is a flowchart illustrating the method of generating the bleeding point identification according to an embodiment of the present disclosure.

FIG. 5B is a schematic diagram illustrating the surgical image set with the bleeding point identification according to an embodiment of the present disclosure.

FIG. 6A is a flowchart illustrating the method of generating the blood flow path indication according to an embodiment of the present disclosure.

FIG. 6B is a schematic diagram illustrating the surgical image set with the blood flow path indication according to an embodiment of the present disclosure.

FIG. 7 is a flowchart illustrating the method of generating the prompting bleeding point according to an embodiment of the present disclosure.

FIG. 8 is a flowchart illustrating the method of generating the bleeding amount indication according to an embodiment of the present disclosure.

FIG. 9 is a flowchart illustrating the method of generating the bleeding speed indication according to an embodiment of the present disclosure.

FIG. 10A is a flowchart illustrating another method of generating surgical condition information according to an embodiment of the present disclosure.

FIG. 10B is a schematic diagram illustrating one of the surgical image set with the surgical condition information and one of the infrared image set with the surgical condition information according to an embodiment of the present disclosure.

### DETAILED DESCRIPTION OF THE INVENTION

To facilitate understanding of the object, characteristics and effects of this present disclosure, embodiments together with the attached drawings for the detailed description of the present disclosure are provided.

Before the present disclosure is described in detail, it should be noted that the same components or steps may be denoted by the same reference numeral in the following description.

It should be also noted that in the context of the present disclosure, the terms such as “first”, “second”, “third”, and “fourth” are used to differentiate between components instead of being not used to limit the components themselves or to indicate a particular ordering of the components.

It should be also noted that the various steps described herein may be performed in sequential, in reverse order, or by appropriately changing or skipping the step during controlling and processing.

It should be also noted that the phrase “the first step may be performed in sequence after the second step is performed” may represent that after the second step is performed, the first step may be performed directly or be performed after performing another step (e.g., the third step) first.

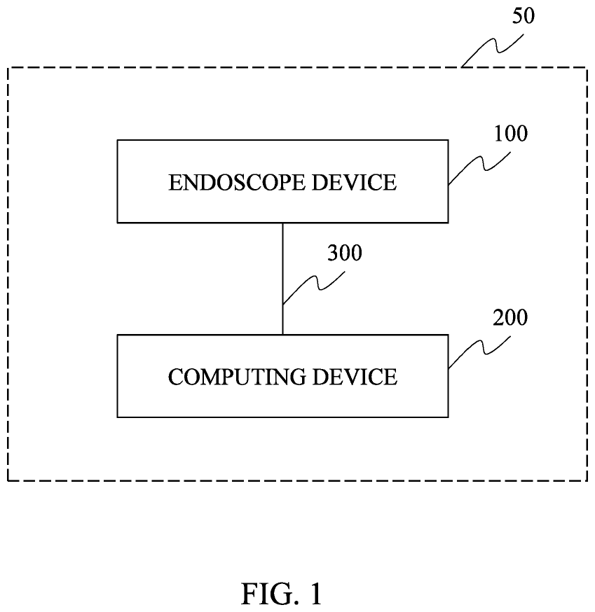

Referring to FIG. 1, FIG. 1 is a schematic diagram illustrating a system 50 for generating surgical condition information according to an embodiment of the present disclosure. In some embodiments, the system 50 includes an endoscope device 100, a computing device 200, and a signal transmitting path 300.

In some embodiments, the endoscope device 100 signally connects with the computing device 200 via the signal transmitting path 300. That is, the signal transmitting path 300 may be configured to transmit the signal (such as images and/or data) between the endoscope device 100 and the computing device 200. In some embodiments, the signal transmitting path 300 is a physical transmission line. In some embodiments, the signal transmitting path 300 is a virtual transmission line.

In some embodiments, the endoscope device 100 is configured to photograph a surgical image set of a target object immediately and is configured to transmit the photographed surgical image set to the computing device 200 and/or display the photographed surgical image set on a displaying device. In a particular example, the endoscope device 100 may include one or more processors and may perform various functions (for example, photographing images, and/or sending or receiving images, data, and/or instructions) through hardware-software synergy. The endoscope device 100 may also be provided in the form of any type of medical image devices as needed. That is, the endoscope device 100 described in the present disclosure may be any device that can photograph and transmit the surgical image set.

In some embodiments, the endoscope device 100 further includes a beam splitter such that the endoscope device 100 is able to photograph both the surgical image set and the infrared image set, wherein the surgical image set contains at least one image with a visible light feature and the infrared image set contains at least one image with an infrared feature. That is, the surgical image set is photographed in the presence of a visible light by the endoscope device 100 and the infrared image set is photographed in the presence of an infrared light by the endoscope device 100.

Therefore, the endoscope device 100 can photograph the surgical image set and the infrared image set with the different wavelengths of lights. The endoscope device 100 can generate the different wavelengths of lights. Further, the endoscope device 100 can filter out or select the different wavelengths of lights to form a specific wavelength range image.

In some embodiments, the computing device 200 is configured to execute specific codes, instructions, and/or algorithms and is configured to receive the surgical image set from the endoscope device 100 and perform the steps for the surgical image set. For example, the computer device 200 may be a computer, a server, a laptop, a mobile device or any other device that is capable of performing data computation and/or performing data and/or image processing, but the present disclosure is not limited thereto.

In some embodiments, the system 50 further includes a displaying device (not shown in FIG. 1). In some embodiments, the displaying device is configured to signally connect with the endoscope device 100 and/or the computing device 200 and is configured to display the photographed surgical image set from the endoscope device 100 and/or display the images and/or the data processed by the computing device 200. For example, the displaying device may be a computer screen, a medical image display device or any other device that is capable of displaying images and/or data, but the present disclosure is not limited thereto. In some embodiments, the displaying device is configured to display the surgical image set with the surgical condition information.

Based on the above, the endoscope device 100 can immediately photograph the surgical image set of the target object and can transmit the photographed surgical image set to the computing device 200 via the signal transmitting path 300 such that the computing device 200 can receive the photographed surgical image set and can generate the surgical condition information based on the received surgical image set.

Due to the system 50 as shown in FIG. 1, the present disclosure can contribute to the technical field about processing and analyzing the surgical image set.

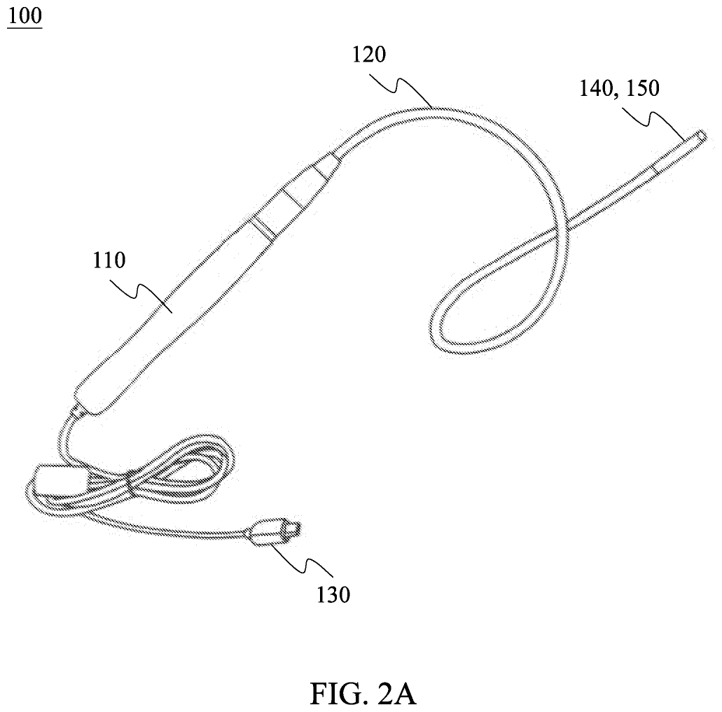

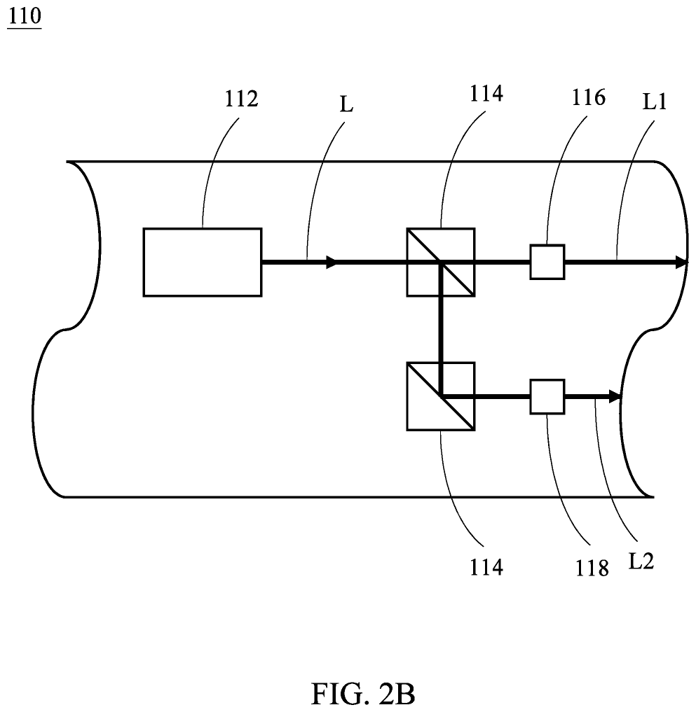

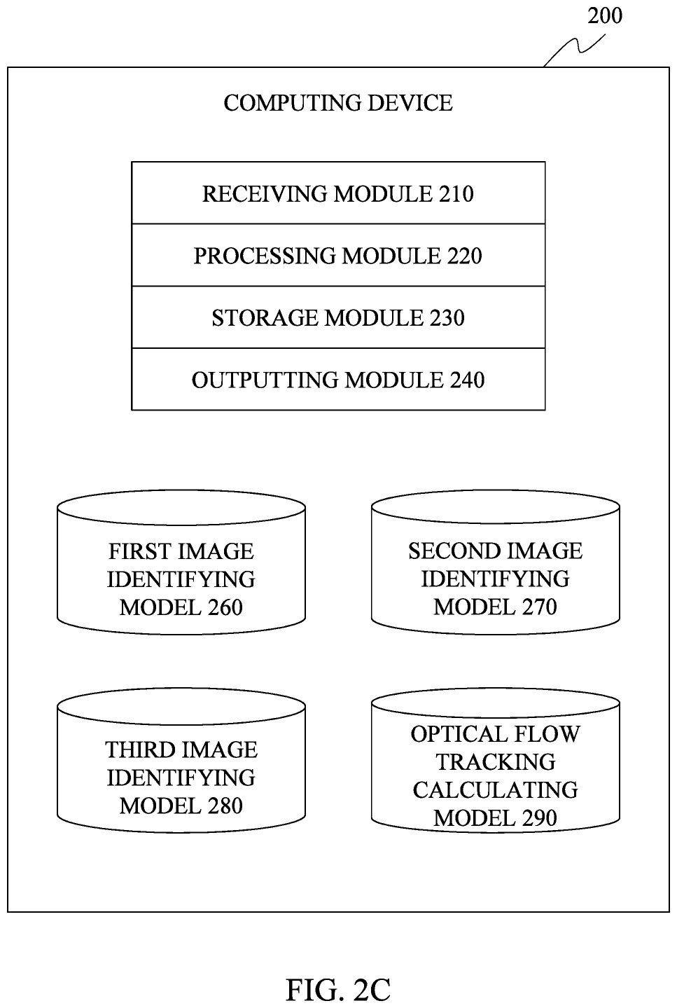

Referring to both FIG. 2A and FIG. 2B. FIG. 2A is a perspective view of the endoscope device 100 as shown in FIG. 1 according to an embodiment of the present disclosure. FIG. 2B is a schematic diagram illustrating internal components of the main body 110 as shown in FIG. 2A according to an embodiment of the present disclosure. Specifically, the endoscope device 100 in this embodiment includes a main body 110, a catheter 120, and a cable 130. The catheter 120 is configured to connect with the main body 110 and is configured to be flexible for enveloping an endoscopic camera and for transmitting a light that is needed for photographing the images. The cable 130 is configured to connect with the main body 110 to transmit the electricity and/or the data to or from the computing device 200. However, in other embodiments, the endoscope device 100 may also be chargeable and implement wireless signal connection with the computing device 200.

In addition, the endoscope device 100 may further include a first detecting unit 140 and a second detecting unit 150 that are disposed at distal of the catheter 120. The first detecting unit 140 is adapted to capture the visible light, and the second detecting unit 150 is adapted to capture the special light such as an infrared ray or a narrow band light. The first detecting unit 140 and the second detecting unit 150 may be two separate parts or two portions of a single component. In other words, the endoscope device 100 is capable of detecting and/or photographing the images resulted from the visible light and the special light individually or simultaneously.

As shown in FIG. 2B, the main body 110 may accommodate a light source 112, at least one light splitting member 114, a first light gate 116, and a second light gate 118. The light source 112 can emit a single light beam L and project the single light beam L to the light splitting member 114. The light splitting member 114, such as lenses or prisms, is capable of splitting the light beam L into a visible light L1 and a special light L2 such as an infrared ray or a narrow band light. The first light gate 116 and the second light gate 118 respectively control passing of the passages of the visible light L1 and the special light L2. Therefore, the endoscope device 100 can realize projecting two different wavelengths of lights by providing a single light source and reduce occupied volume. However, in other embodiments, the main body 110 may also include one visible light source and one special light, and the endoscope device 100 selectively projects the visible light source and/or the special light by operations of corresponding light sources.

Referring to FIG. 2C, FIG. 2C is a schematic diagram illustrating the computing device 200 as shown in FIG. 1 according to an embodiment of the present disclosure. In some embodiments, the computing device 200 includes a receiving module 210, a processing module 220, and a storage module 230. In some embodiments, the computing device 200 further includes a first image identifying model 260, a second image identifying model 270, a third image identifying model 280, and/or an optical flow tracking calculating model 290. That is, the first image identifying model 260, the second image identifying model 270, the third image identifying model 280, and/or the optical flow tracking calculating model 290 may be integrated into the computing device 200 depending on the user and/or the designer.

In some embodiments, the receiving module 210 is configured to signally connect with the endoscope device 100 and is configured to receive the images (such as the surgical image set and/or the infrared image set) and/or the data (such as the information about the received images).

In some embodiments, the computing device 200 stores the received images and/or the received data into a database (not shown in FIG. 1). In some embodiments, the database is a cloud server, but the present disclosure is not limited thereto.

In some embodiments, the storage module 230 is configured to store codes, instructions, and/or algorithms. In some embodiments, the storage module 230 includes one or more non-volatile memories and/or one or more volatile memories. In some embodiments, the non-volatile memory is, for example, read-only memory, flash memory, or non-volatile random access memory, but the present disclosure is not limited thereto. In some embodiments, the volatile memory is, for example, dynamic random access memory or static random access memory, but the present disclosure is not limited thereto. Since the storage module 230 can store the specific codes, instructions, and/or algorithms, the computing device 200 can execute the specific codes, instructions, and/or algorithms in order to perform the specific steps.

In some embodiments, the processing module 220 is configured to signally connect with the receiving module 210 and the storage module 230. That is, the processing module 220 can receive the images and/or the data through the receiving module 210 and can read and/or execute the codes, instructions, and/or algorithms, that are stored in the storage module 230. In some embodiments, the processing module 220 is, for example, a central processing unit or a graphics processing unit, but the present disclosure is not limited thereto. The processing module 220 can perform the specific steps after the processing module 220 loads and executes the specific codes, instructions, and/or algorithms. In particular, the processing module 220 can perform the steps mentioned in the methods of generating surgical condition information such that the processing module 220 can automatically generate the surgical condition information based on the surgical image set. Therefore, the processing module 220 as shown in FIG. 2C can contribute to the technical field about processing and analyzing the surgical image set.

In some embodiments, the first image identifying model 260 is configured to identify the images, such as the surgical image set. That is, the first image identifying model 260 may identify the images after the images are input into the first image identifying model 260. In some embodiments, the first image identifying model 260 may further generate a first identified result (such as the bleeding warning) and output the first identified result. In some embodiments, the first image identifying model 260 may be, for example, a DenseNet model, a VGG model, an efficientnet model, an AlexNet model, a ConvNext model, and/or a GoogLeNet model, but the present disclosure is not limited thereto.

In addition, the first image identifying model 260 has been trained by plural pieces of first training images in advance. In some embodiments, the first training images may be photographed by the endoscope device 100. In some embodiments, each of the first training images may further contain a first pre-identified result that is either with bleeding or without bleeding. In some embodiments, the first pre-identified result may be identified by a professional, such as a medical professional or surgeon. In some embodiments, the first pre-identified result may be determined by whether the blood area occupies more than fifty percent of the first training image and/or whether the bleed point exists in the first training image. That is, the first training image contains the first pre-identified result with bleed when the blood area occupies more than fifty percent of the first training image and/or the bleed point exists in the first training image, and vice versa. In some embodiments, the first training image may be divided into a smaller size. In some embodiments, the first training image may be standardized to the same size. That is, in some embodiments, the first training images may be divided and/or standardized before the first training images are used to train the first image identifying model 260.

Since the first image identifying model 260 has been trained by plural pieces of the first training images in advance, the first image identifying model 260 can be used to identify a new surgical image set in a first prediction accuracy rate. In some embodiments, the first prediction accuracy rate of the first image identifying model 260 may be designed and validated as 0.8 or more. Preferably, the first prediction accuracy rate of the first image identifying model 260 may be designed and validated as 0.95 or more. Therefore, a new surgical image set that is input into the first image identifying model 260 may be accurately identified whether the new surgical image set is either with bleeding or without bleeding by the first image identifying model 260 that has been trained in advance.

In some embodiments, the first image identifying model 260 can determine whether the bleeding exists in the surgical image set by extracting the features from the surgical image set and then analyzing the extracted features. The features that are extracted from the surgical image set may be the variation in the pixels of the adjacent images of the surgical image set, e.g., the variation in the location of the red pixels, the brightness of the red pixels, and/or the numbers of the red pixels.

In some embodiments, the second image identifying model 270 is configured to identify the images, such as the surgical image set. That is, the second image identifying model 270 may identify the images after the images are input into the second image identifying model 270. In some embodiments, the second image identifying model 270 may further generate a second identified result (such as the bleeding point identification) and output the second identified result. In some embodiments, the second image identifying model 270 may be, for example, an R-CNN model, a Fast R-CNN model, a Faster R-CNN model, a Mask R-CNN model, a YOLO model and/or a Unet model, but the present disclosure is not limited thereto.

In addition, the second image identifying model 270 has been trained by plural pieces of second training images in advance. In some embodiments, the second training images may be photographed by the endoscope device 100. In some embodiments, each of the second training images may further contain a second pre-identified result, e.g., at least one bleeding point. In some embodiments, the second pre-identified result may be identified and/or be marked on the corresponding second training image by a professional, such as a medical professional or surgeon. In some embodiments, the second training image may be divided into a smaller size. In some embodiments, the second training image may be standardized to the same size. That is, in some embodiments, the second training images may be divided and/or standardized before the second training images are used to train the second image identifying model 270.

Since the second image identifying model 270 has been trained by plural pieces of the second training images in advance, the second image identifying model 270 can be used to identify a new surgical image set in a second prediction accuracy rate. In some embodiments, the second prediction accuracy rate of the second image identifying model 270 may be designed and validated as 0.8 or more. Preferably, the second prediction accuracy rate of the second image identifying model 270 may be designed and validated as 0.9 or more. Therefore, a new surgical image set that is input into the second image identifying model 270 may be accurately identified whether the at least one bleeding point exists or not and/or where the at least one bleeding point is by the second image identifying model 270 that has been trained in advance.

In some embodiments, the second image identifying model 270 can determine whether the bleeding exists in the surgical image set by extracting the features from the surgical image set and then analyzing the extracted features. The features that are extracted from the surgical image set may be the variation in the pixels of the adjacent images of the surgical image set, e.g., the variation in the location of the red pixels, the brightness of the red pixels, and/or the numbers of the red pixels.

In some embodiments, the third image identifying model 280 is configured to identify the images, such as the surgical image set. That is, the third image identifying model 280 may identify the images after the images are input into the third image identifying model 280. In some embodiments, the third image identifying model 280 may further generate a third identified result (such as the bleeding area indication) and output the third identified result. In some embodiments, the third image identifying model 280 may be, for example, a Mask R-CNN model, a YOLO model and/or a Unet model, but the present disclosure is not limited thereto.

In addition, the third image identifying model 280 has been trained by plural pieces of third training images in advance. In some embodiments, the third training images may be photographed by the endoscope device 100. In some embodiments, each of the third training images may further contain a third pre-identified result, i.e., the bleeding area indication. In some embodiments, the third pre-identified result may be identified and/or be marked on the corresponding third training image by a professional, such as a medical professional or surgeon. In some embodiments, the third training image may be divided into a smaller size. In some embodiments, the third training image may be standardized to the same size. That is, in some embodiments, the third training images may be divided and/or standardized before the third training images are used to train the third image identifying model 280.

Since the third image identifying model 280 has been trained by plural pieces of the third training images in advance, the third image identifying model 280 can be used to identify a new surgical image set in a third prediction accuracy rate. In some embodiments, the third prediction accuracy rate of the third image identifying model 280 may be designed and validated as 0.8 or more. Preferably, the third prediction accuracy rate of the third image identifying model 280 may be designed and validated as 0.9 or more. Therefore, a new surgical image set that is input into the third image identifying model 280 may be accurately identified whether the bleeding area exists or not and/or how much the pixels corresponding to the bleeding area is by the third image identifying model 280 that has been trained in advance.

In some embodiments, the third image identifying model 280 can determine whether the bleeding exists in the surgical image set by extracting the features from the surgical image set and then analyzing the extracted features. The features that are extracted from the surgical image set may be the variation in the pixels of the adjacent images of the surgical image set, e.g., the variation in the location of the red pixels, the brightness of the red pixels, and/or the numbers of the red pixels.

In some embodiments, the optical flow tracking calculating model 290 is configured to calculate the optical flow for the images. That is, the optical flow tracking calculating model 290 may calculate the optical flow for the images after the images are input into the optical flow tracking calculating model 290. In some embodiments, the optical flow tracking calculating model 290 may further generate an optical flow result and output the optical flow result. In particular, the optical flow tracking calculating model 290 may calculate the optical flow for the surgical image set that are input into the optical flow tracking calculating model 290. In some embodiments, the optical flow tracking calculating model 290 may further generate the blood flow path indication and output the blood flow path indication.

In some embodiments, the optical flow tracking calculating model 290 may be implemented, for example, by the Lucas-Kanade method, the Gunnar-Farneback optical flow, the block matching method, the Horn-Schunck method, and/or the SimpleFlow, but the present disclosure is not limited thereto.

In some embodiments, the computing device 200 may further include an outputting module 240. That is, the outputting module 240 may be integrated into the computing device 200 depending on the user and/or the designer. In some embodiments, the outputting module 240 is configured to output the surgical condition information to the displaying device and/or the server.

Based on the above, the present disclosure can generate the surgical condition information based on the received surgical image set by the computing device 200 as shown in FIG. 2C. Besides, the computing device 200 as shown in FIG. 2C can contribute to the technical field about processing and analyzing the surgical image set.

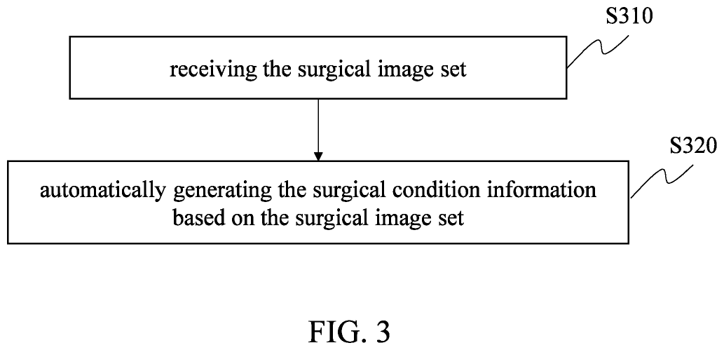

Referring to FIG. 3, FIG. 3 is a flowchart illustrating a method of generating surgical condition information according to an embodiment of the present disclosure. In some embodiments, the method of generating surgical condition information as shown in FIG. 3 may be implemented by the computing device as shown in FIG. 2C. In some embodiments, the method of generating surgical condition information may include the steps S310 and S320.

In some embodiments, the step S310 may be performed by the processing module as shown in FIG. 2C. In the step S310, the processing module may receive the surgical image set through the receiving module as shown in FIG. 2C.

In some embodiments, the step S320 may be performed by the processing module as shown in FIG. 2C. In some embodiments, the step S320 may be performed in sequence after the step S310 is performed. In the step S320, the processing module may automatically generate the surgical condition information based on the surgical image set. In some embodiments, the surgical condition information may contain the bleeding warning, the bleeding point identification, the blood flow path indication, the prompting bleeding point, the bleeding amount indication, and/or the bleeding speed indication.

In particular, in some embodiments, the processing module may automatically generate the bleeding warning corresponding to the received surgical image set by utilizing the first image identifying model as shown in FIG. 2C after the step S320 is performed by the processing module. In particular, in some embodiments, the processing module may automatically generate the bleeding point identification corresponding to the received surgical image set by utilizing the second image identifying model as shown in FIG. 2C after the step S320 is performed by the processing module. In particular, in some embodiments, the processing module may automatically generate the blood flow path indication corresponding to the received surgical image set by utilizing the optical flow tracking calculating model as shown in FIG. 2C after the step S320 is performed by the processing module. In particular, in some embodiments, the processing module may automatically generate the prompting bleeding point based on the blood flow path indication that is generated by utilizing the optical flow tracking calculating model as shown in FIG. 2C after the step S320 is performed by the processing module. In particular, in some embodiments, the processing module may automatically generate the bleeding amount indication based on the bleeding area indication that is generated by utilizing the third image identifying model as shown in FIG. 2C after the step S320 is performed by the processing module. In particular, in some embodiments, the processing module may automatically generate the bleeding speed indication based on the bleeding amount indication after the step S320 is performed by the processing module.

Based on the above, the present disclosure can generate the surgical condition information based on the received surgical image set by the method as shown in FIG. 3 such that the user can be aware of the bleeding status of the target object and decide on an appropriate procedure for coping with the internal bleeding. Besides, the method as shown in FIG. 3 can contribute to the technical field about processing and analyzing the surgical image set.

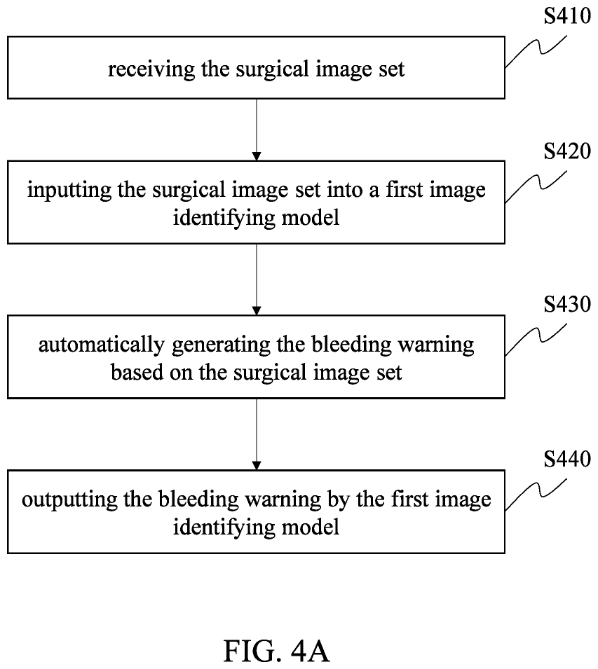

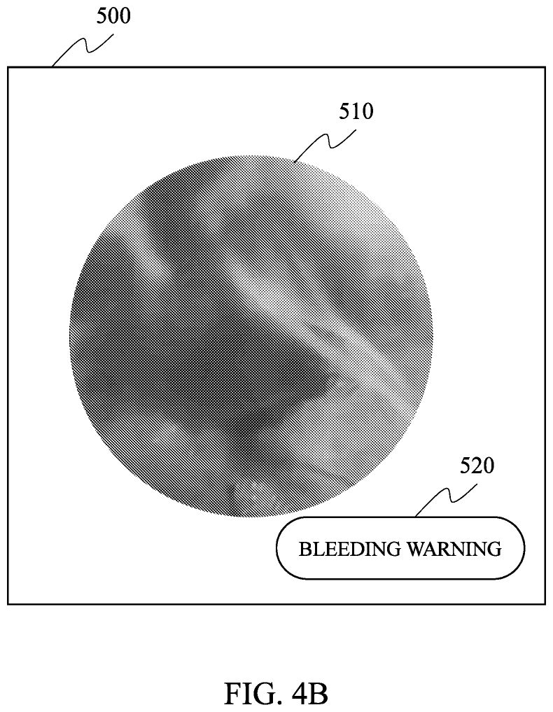

Referring to FIG. 4A, FIG. 4A is a flowchart illustrating the method of generating the bleeding warning according to an embodiment of the present disclosure. In some embodiments, the method of generating surgical condition information as shown in FIG. 4A may be implemented by the computing device as shown in FIG. 2C. In some embodiments, the method of generating surgical condition information may include the steps S410, S420, S430, and S440, wherein the step S410 is substantially the same as the step S310 as shown in FIG. 3.

In some embodiments, the step S420 may be performed by the processing module as shown in FIG. 2C. In some embodiments, the step S420 may be performed in sequence after the step S410 is performed. In the step S420, the processing module may input the surgical image set into the first image identifying model as shown in FIG. 2C.

In some embodiments, the step S430 may be performed by the processing module as shown in FIG. 2C. In some embodiments, the step S430 may be performed in sequence after the step S420 is performed. In the step S430, the processing module may automatically generate the bleeding warning based on the surgical image set by the first image identifying model.

Since the first image identifying model has been trained by plural pieces of the first training images in advance, the first image identifying model can be used in the step S430 in order to identify the surgical image set and generate the bleeding warning corresponding to the surgical image set. In particular, the first image identifying model may analyze the feature of the surgical image set to determine whether the blood area occupies more than fifty percent of the surgical image set and/or whether the bleed point exists in the surgical image set such that the first image identifying model can generate the bleeding warning based on the surgical image set.

In some embodiments, the first image identifying model that is used in the step S430 may be, for example, a DenseNet model, a VGG model, an efficientnet model, an AlexNet model, a ConvNext model, and/or a GoogLeNet model, but the present disclosure is not limited thereto. In particular, the first prediction accuracy rate of the first image identifying model may reach 0.95 or more when the efficientnet model is used as the first image identifying model in the step S430.

In some embodiments, the step S440 may be performed by the processing module as shown in FIG. 2C. In some embodiments, the step S440 may be performed in sequence after the step S430 is performed. In the step S440, the processing module may output the bleeding warning by the first image identifying model.

Based on the above, the present disclosure can generate the surgical condition information, i.e., the bleeding warning, based on the received surgical image set by the method as shown in FIG. 4A such that the user can be aware of the bleeding status of the target object and decide on an appropriate procedure for coping with the internal bleeding. Besides, the method as shown in FIG. 4A can contribute to the technical field about processing and analyzing the surgical image set.

Referring to FIG. 4B, FIG. 4B is a schematic diagram illustrating the surgical image set 510 with the bleeding warning 520 according to an embodiment of the present disclosure. FIG. 4B is a colored figure.

In some embodiments, the surgical image set 510 may be photographed by the endoscope device as shown in FIG. 2A. Since the computing device as shown in FIG. 2C may generate the bleeding warning 520 based on the surgical image set 510, the flag that represents the bleeding warning 520 may be set as either a high level or a low level depends on the first identified result that is identified by the first image identifying model as shown in FIG. 2C. In particular, the flag may be set as a high level when the first identified result is with bleeding, and vice versa. In some embodiments, the bleeding warning 520 can be displayed with the surgical image set 510 synchronously in a displaying region 500.

Based on the above, the present disclosure can clearly display the bleeding warning 520 with the surgical image set 510 in the displaying region 500 such that the user can be immediately aware of the bleeding status of the target object and decide on an appropriate procedure for coping with the internal bleeding.

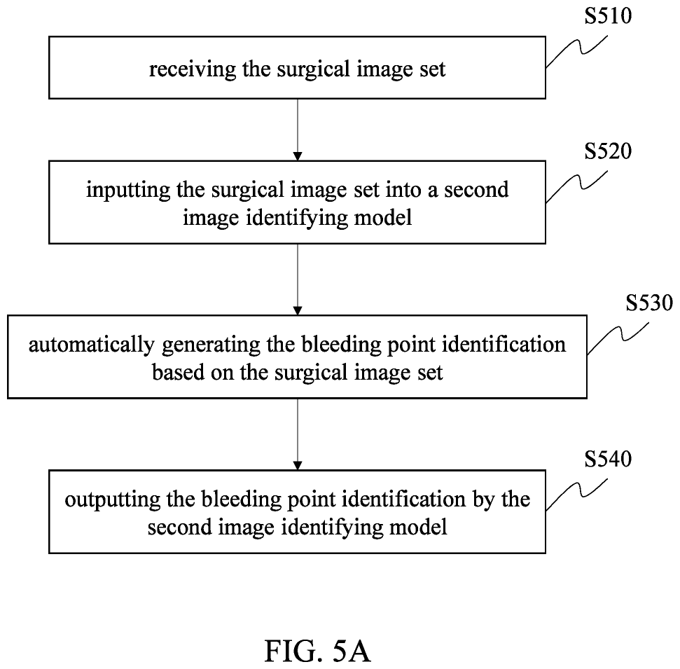

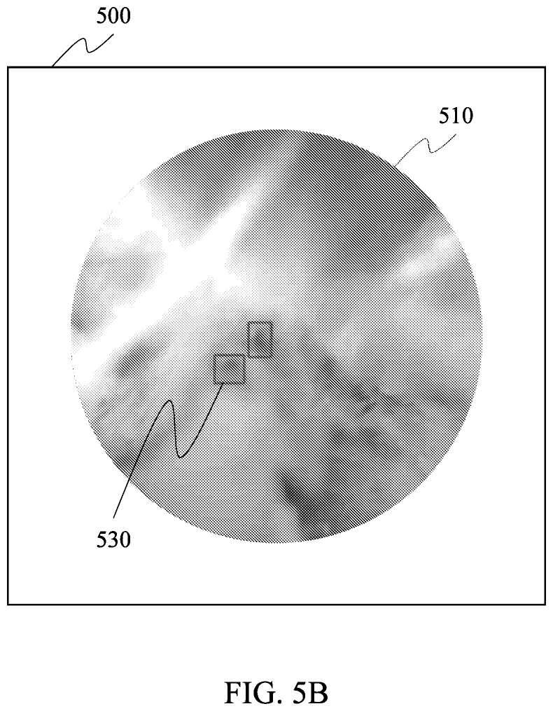

Referring to FIG. 5A, FIG. 5A is a flowchart illustrating the method of generating the bleeding point identification according to an embodiment of the present disclosure. In some embodiments, the method of generating surgical condition information as shown in FIG. 5A may be implemented by the computing device as shown in FIG. 2C. In some embodiments, the method of generating surgical condition information may include the steps S510, S520, S530, and S540, wherein the step S510 is substantially the same as the step S310 as shown in FIG. 3.

In some embodiments, the step S520 may be performed by the processing module as shown in FIG. 2C. In some embodiments, the step S520 may be performed in sequence after the step S510 is performed. In the step S520, the processing module may input the surgical image set into the second image identifying model as shown in FIG. 2C.

In some embodiments, the step S530 may be performed by the processing module as shown in FIG. 2C. In some embodiments, the step S530 may be performed in sequence after the step S520 is performed. In the step S530, the processing module may automatically generate the bleeding point identification based on the surgical image set by the second image identifying model.

Since the second image identifying model has been trained by plural pieces of the second training images in advance, the second image identifying model can be used in the step S530 in order to identify the surgical image set and generate the bleeding point identification corresponding to the surgical image set. In particular, the second image identifying model may analyze the feature of the surgical image set to determine whether the at least one bleeding point exists or not and/or where the at least one bleeding point is such that the second image identifying model can generate the bleeding point identification based on the surgical image set.

In some embodiments, the second image identifying model that is used in the step S530 may be, for example, an R-CNN model, a Fast R-CNN model, a Faster R-CNN model, a Mask R-CNN model, a YOLO model and/or a Unet model, but the present disclosure is not limited thereto. In particular, the second prediction accuracy rate of the second image identifying model may reach 0.9 or more when the YOLOv8 model is used as the second image identifying model in the step S530.

In some embodiments, the step S540 may be performed by the processing module as shown in FIG. 2C. In some embodiments, the step S540 may be performed in sequence after the step S530 is performed. In the step S540, the processing module may output the bleeding point identification by the second image identifying model.

Based on the above, the present disclosure can generate the surgical condition information, i.e., the bleeding point identification, based on the received surgical image set by the method as shown in FIG. 5A such that the user can be aware of the bleeding status of the target object and decide on an appropriate procedure for coping with the internal bleeding. Besides, the method as shown in FIG. 5A can contribute to the technical field about processing and analyzing the surgical image set.

Referring to FIG. 5B, FIG. 5B is a schematic diagram illustrating the surgical image set with the bleeding point identification according to an embodiment of the present disclosure. FIG. 5B is a colored figure.

In some embodiments, the surgical image set 510 may be photographed by the endoscope device as shown in FIG. 2A and the bleeding point identification 530 may be generated by the computing device as shown in FIG. 2C. In some embodiments, the bleeding point identification 530 may be a coordinate information that indicates the position of the bleeding point in the 2D coordinate plane. In some embodiments, the bleeding point identification 530 may be an icon that be marked on the surgical image set. In some embodiments, the bleeding point identification 530 can be displayed with the surgical image set 510 synchronously in a displaying region 500.

Based on the above, the present disclosure can clearly display the bleeding point identification 530 with the surgical image set 510 in the displaying region 500 such that the user can be immediately aware of the bleeding status of the target object and decide on an appropriate procedure for coping with the internal bleeding.

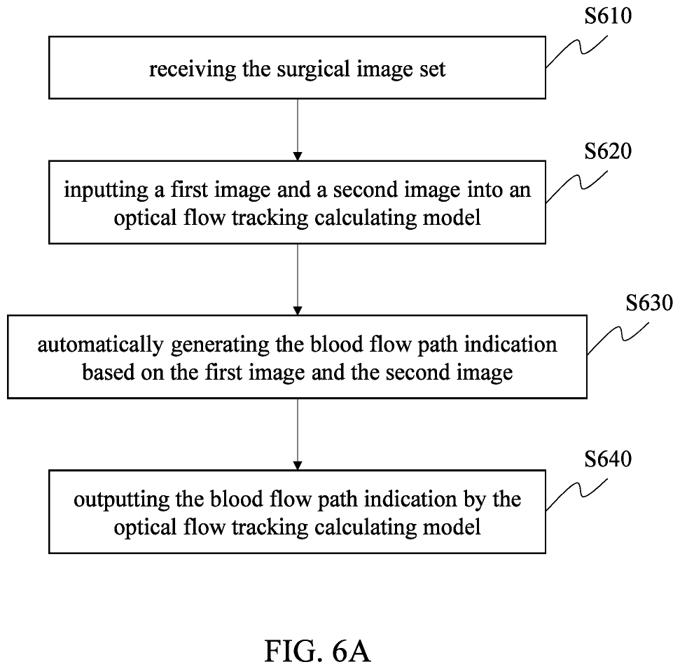

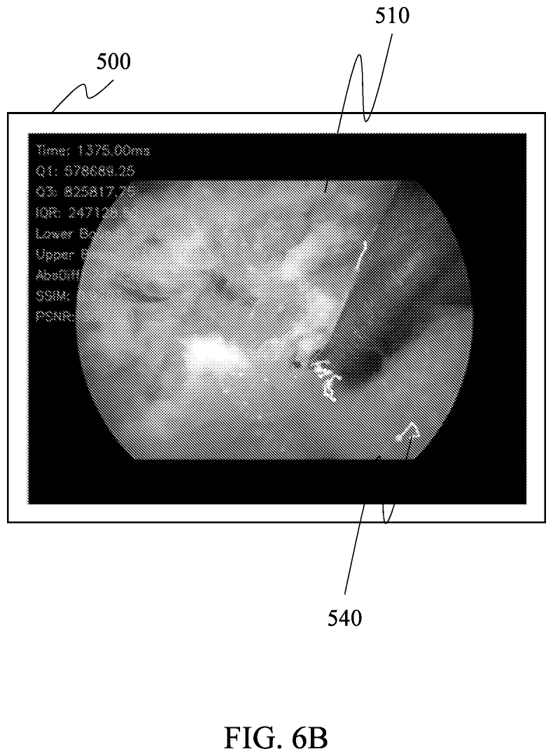

Referring to FIG. 6A, FIG. 6A is a flowchart illustrating the method of generating the blood flow path indication according to an embodiment of the present disclosure. In some embodiments, the method of generating surgical condition information as shown in FIG. 6A may be implemented by the computing device as shown in FIG. 2C. In some embodiments, the method of generating surgical condition information may include the steps S610, S620, S630, and S640, wherein the step S610 is substantially the same as the step S310 as shown in FIG. 3.

In some embodiments, the surgical image set that are received through the receiving module as shown in FIG. 2C may further contain a first image and a second image. The first image and the second image may be images that are continuously photographed by the endoscope device as shown in FIG. 2A. The first image and the second image may display at least one same region.

In some embodiments, the step S620 may be performed by the processing module as shown in FIG. 2C. In some embodiments, the step S620 may be performed in sequence after the step S610 is performed. In the step S620, the processing module may input the first image and the second image into the optical flow tracking calculating model as shown in FIG. 2C.

In some embodiments, the step S630 may be performed by the processing module as shown in FIG. 2C. In some embodiments, the step S630 may be performed in sequence after the step S620 is performed. In the step S630, the processing module may automatically generate the blood flow path indication based on the first image and the second image by the optical flow tracking calculating model.

Since the optical flow tracking calculating model can analyze and calculate the difference (such as the brightness change of the pixels) between the first image and the second image, the optical flow tracking calculating model can generate the optical flow, i.e., the blood flow path indication. In some embodiments, the optical flow tracking calculating model may be implemented, for example, by the Lucas-Kanade method, the Gunnar-Farneback optical flow, the block matching method, the Horn-Schunck method, and/or the SimpleFlow, but the present disclosure is not limited thereto.

In some embodiments, the step S640 may be performed by the processing module as shown in FIG. 2C. In some embodiments, the step S640 may be performed in sequence after the step S630 is performed. In the step S640, the processing module may output the blood flow path indication by the optical flow tracking calculating model.

Based on the above, the present disclosure can generate the surgical condition information, i.e., the blood flow path indication, based on the received surgical image set by the method as shown in FIG. 6A such that the user can be aware of the bleeding status of the target object and decide on an appropriate procedure for coping with the internal bleeding. Besides, the method as shown in FIG. 6A can contribute to the technical field about processing and analyzing the surgical image set.

Referring to FIG. 6B, FIG. 6B is a schematic diagram illustrating the surgical image set with the blood flow path indication according to an embodiment of the present disclosure. FIG. 6B is a colored figure.

In some embodiments, the surgical image set 510 may be photographed by the endoscope device as shown in FIG. 2A and the blood flow path indication 540 may be generated by the computing device as shown in FIG. 2C. In some embodiments, the blood flow path indication 540 may be an icon that be marked on the surgical image set. In some embodiments, the blood flow path indication 540 can be displayed with the surgical image set 510 synchronously in a displaying region 500.

Based on the above, the present disclosure can clearly display the blood flow path indication 540 with the surgical image set 510 in the displaying region 500 such that the user can be immediately aware of the bleeding status of the target object and decide on an appropriate procedure for coping with the internal bleeding.

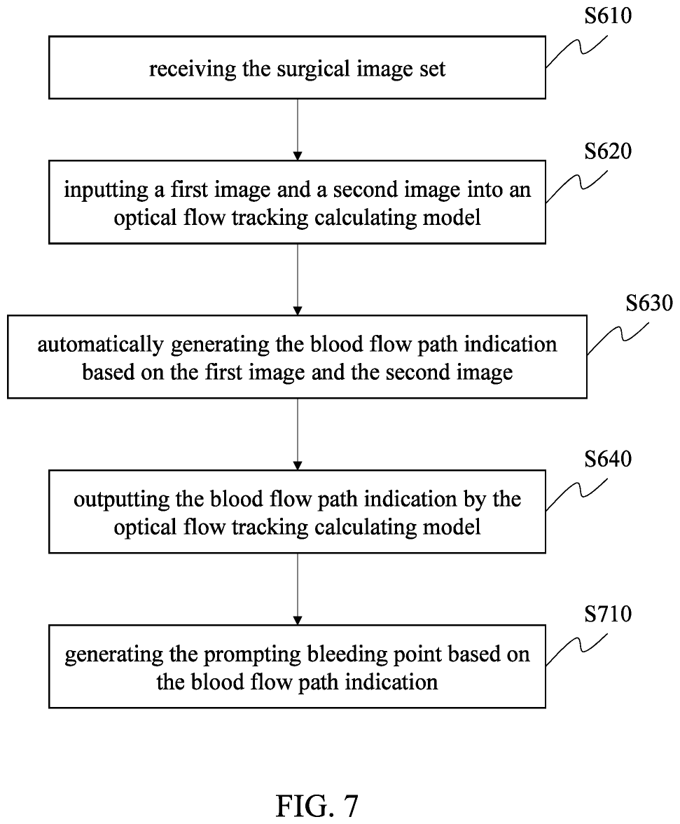

Referring to FIG. 7, FIG. 7 is a flowchart illustrating the method of generating the prompting bleeding point according to an embodiment of the present disclosure. In some embodiments, the method of generating surgical condition information as shown in FIG. 7 may be implemented by the computing device as shown in FIG. 2C. In some embodiments, the method of generating surgical condition information may include the steps S610, S620, S630, S640, and S710, wherein the steps S610, S620, S630, and S640 are substantially the same as the method as shown in FIG. 6A.

In some embodiments, the step S710 may be performed by the processing module as shown in FIG. 2C. In some embodiments, the step S710 may be performed in sequence after the step S640 is performed. In the step S710, the processing module may generate the prompting bleeding point based on the blood flow path indication. In some embodiments, since the optical flow tracking calculating model may further derive the direction of the optical flow, the direction of the blood flow path indication may be derived. Due to the direction of the blood flow path indication, the processing module may generate the prompting bleeding point. In particular, if the direction of the blood flow path indication indicates that the blood flow is from the top of the surgical image set, then the processing module may generate the prompting bleeding point, such as an information that indicates the user to move up. In some embodiments, the prompting bleeding point can be displayed with the surgical image set synchronously in a displaying region.

Based on the above, the present disclosure can generate the surgical condition information, i.e., the prompting bleeding point, based on the received surgical image set by the method as shown in FIG. 7 such that the user can be aware of the bleeding status of the target object and decide on an appropriate procedure for coping with the internal bleeding. Besides, the method as shown in FIG. 7 can contribute to the technical field about processing and analyzing the surgical image set.

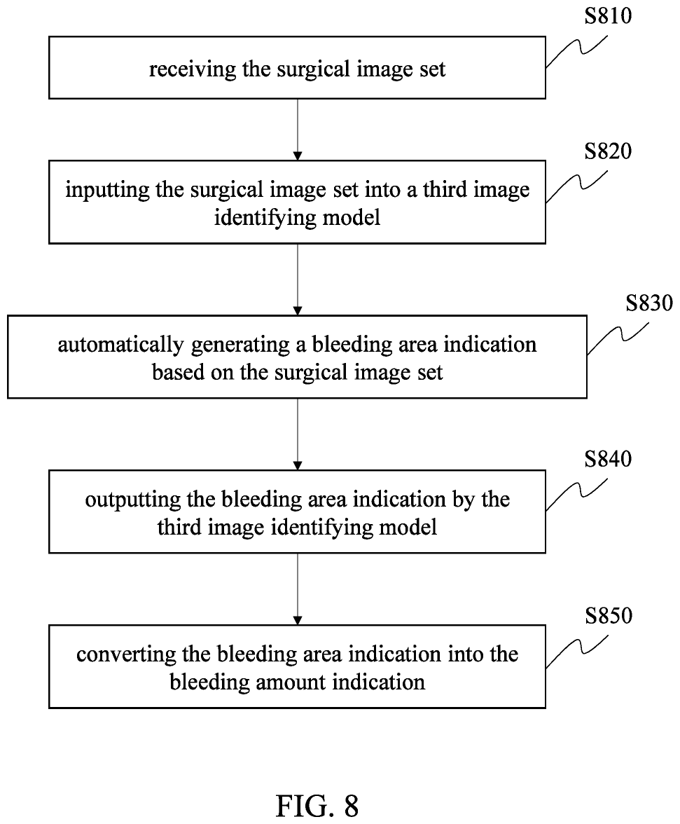

Referring to FIG. 8, FIG. 8 is a flowchart illustrating the method of generating the bleeding amount indication according to an embodiment of the present disclosure. In some embodiments, the method of generating surgical condition information as shown in FIG. 8 may be implemented by the computing device as shown in FIG. 2C. In some embodiments, the method of generating surgical condition information may include the steps S810, S820, S830, S840 and S850, wherein the step S810 is substantially the same as the step S310 as shown in FIG. 3.

In some embodiments, the step S820 may be performed by the processing module as shown in FIG. 2C. In some embodiments, the step S820 may be performed in sequence after the step S810 is performed. In the step S820, the processing module may input the surgical image set into the third image identifying model as shown in FIG. 2C.

In some embodiments, the step S830 may be performed by the processing module as shown in FIG. 2C. In some embodiments, the step S830 may be performed in sequence after the step S820 is performed. In the step S830, the processing module may automatically generate the bleeding area indication based on the surgical image set by the third image identifying model.

Since the third image identifying model has been trained by plural pieces of the third training images in advance, the third image identifying model can be used in the step S830 in order to identify the surgical image set. In particular, the third image identifying model may analyze the feature of the surgical image set to determine whether the bleeding area exists or not, where the bleeding point is, where the boundary of the bleeding area is and/or how much the pixels corresponding to the bleeding area is such that the third image identifying model can generate the bleeding area indication based on the surgical image set.

In some embodiments, the third image identifying model 280 may be, for example, a Mask R-CNN model, a YOLO model and/or a Unet model, but the present disclosure is not limited thereto. In particular, the third prediction accuracy rate of the third image identifying model may reach 0.9 or more when the Unet model is used as the third image identifying model in the step S830.

In some embodiments, the step S840 may be performed by the processing module as shown in FIG. 2C. In some embodiments, the step S840 may be performed in sequence after the step S830 is performed. In the step S840, the processing module may output the bleeding area indication by the third image identifying model.

In some embodiments, the step S850 may be performed by the processing module as shown in FIG. 2C. In some embodiments, the step S850 may be performed in sequence after the step S840 is performed. In the step S850, the processing module may convert the bleeding area indication into the bleeding amount indication. In some embodiments, the bleeding area indication may further contain the pixels corresponding to the bleeding area. In some embodiments, the processing module may convert the pixels corresponding to the bleeding area into the bleeding amount indication by a mapping table. In some embodiments, the bleeding area indication and/or the bleeding amount indication can be displayed with the surgical image set synchronously in a displaying region. In some embodiments, the bleeding area indication and/or the bleeding amount indication can be displayed in a cumulative manner.

Based on the above, the present disclosure can generate the surgical condition information, i.e., the bleeding amount indication, based on the received surgical image set by the method as shown in FIG. 8 such that the user can be aware of the bleeding status of the target object and decide on an appropriate procedure for coping with the internal bleeding. Besides, the method as shown in FIG. 8 can contribute to the technical field about processing and analyzing the surgical image set.

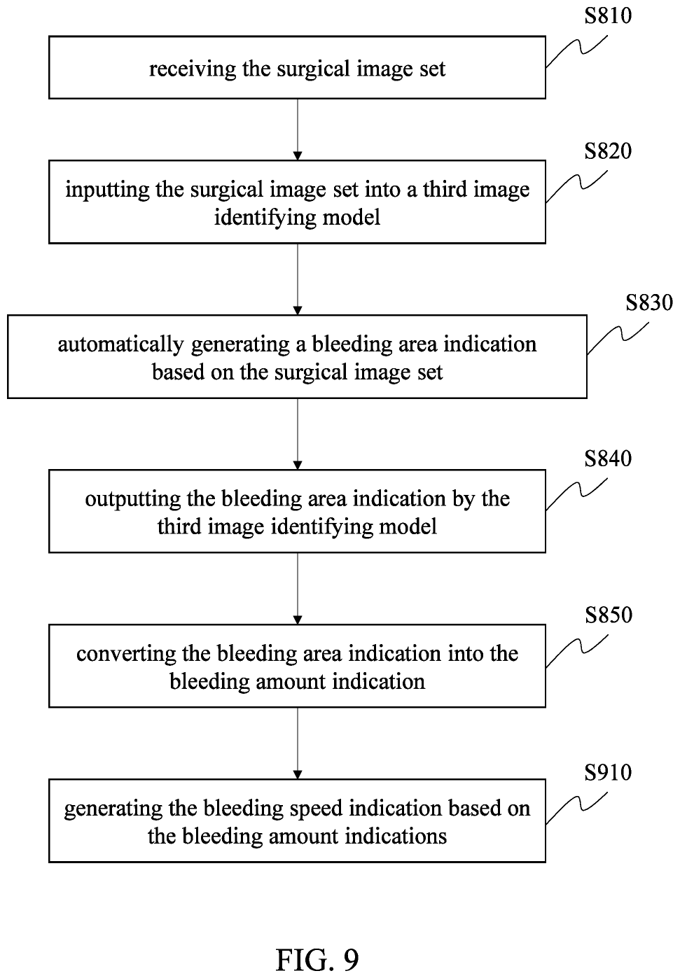

Referring to FIG. 9, FIG. 9 is a flowchart illustrating the method of generating the bleeding speed indication according to an embodiment of the present disclosure. In some embodiments, the method of generating surgical condition information as shown in FIG. 9 may be implemented by the computing device as shown in FIG. 2C. In some embodiments, the method of generating surgical condition information may include the steps S810, S820, S830, S840, S850 and S910, wherein the steps S810, S820, S830, S840, and S850 are substantially the same as the method as shown in FIG. 8.

In some embodiments, the step S910 may be performed by the processing module as shown in FIG. 2C. In some embodiments, the step S910 may be performed in sequence after the step S850 is performed. In the step S910, the processing module may generate the bleeding speed indication based on the bleeding amount indications.

In some embodiments, the surgical image set that are received through the receiving module as shown in FIG. 2C may further contain a third image and a fourth image. The third image and the fourth image may be images that are continuously photographed by the endoscope device as shown in FIG. 2A. The third image and the fourth image may display at least one same region. The bleeding amount indication that is corresponding to the third image and the bleeding amount indication that is corresponding to the fourth image may be generated by the processing module after the step S850 is performed. In some embodiments, the bleeding amount indication may further contain the time when the bleeding amount indication is generated. Since the bleeding amount indication may further contain the time, the processing module may generate the bleeding speed indication by calculating the difference between the bleeding amount indication that is corresponding to the third image and the bleeding amount indication that is corresponding to the fourth image. In some embodiments, the bleeding speed indication can be displayed with the surgical image set synchronously in a displaying region.

Based on the above, the present disclosure can generate the surgical condition information, i.e., the bleeding speed indication, based on the received surgical image set by the method as shown in FIG. 9 such that the user can be aware of the bleeding status of the target object and decide on an appropriate procedure for coping with the internal bleeding. Besides, the method as shown in FIG. 9 can contribute to the technical field about processing and analyzing the surgical image set.

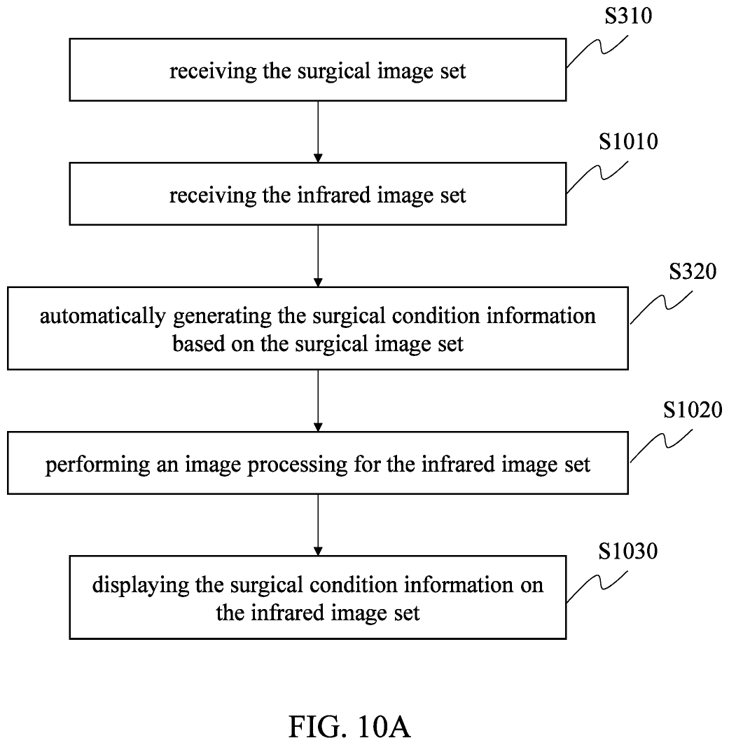

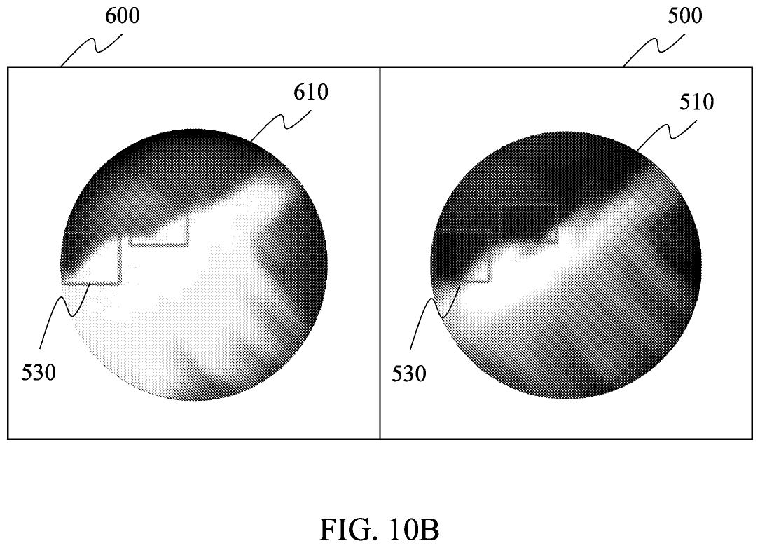

Referring to FIG. 10A, FIG. 10A is a flowchart illustrating another method of generating surgical condition information according to an embodiment of the present disclosure. In some embodiments, the method of generating surgical condition information as shown in FIG. 10A may be implemented by the computing device as shown in FIG. 2C. In some embodiments, the method of generating surgical condition information may include the steps S310, S320, S1010, S1020, and S1030, wherein the steps S310 and S320 are substantially the same as the method as shown in FIG. 3.

In some embodiments, the step S1010 may be performed by the processing module as shown in FIG. 2C. In some embodiments, the step S1010 may be performed with the step S310 synchronously. In the step S1010, the processing module may receive the infrared image set.

In some embodiments, the step S1020 may be performed by the processing module as shown in FIG. 2C. In some embodiments, the step S1020 may be performed in sequence after the step S1010 is performed. In the step S1020, the processing module may perform the image processing for the infrared image set. In some embodiments, the image processing for the infrared image set may refer to reverse the infrared image set and/or rotate the infrared image set. In particular, the received infrared image set may be reverse with y-axis such that the received infrared image set may show the same image as the received surgical image set.

In some embodiments, the step S1030 may be performed by the processing module as shown in FIG. 2C. In some embodiments, the step S1030 may be performed in sequence after the steps S320 and S1020 are performed. In the step S1030, the processing module may display the surgical condition information on the infrared image set. In some embodiments, the surgical condition information may be displayed on the infrared image set and/or the surgical image set synchronously.

Based on the above, the present disclosure can not only generate the surgical condition information based on the received surgical image set but also display the surgical condition information on the infrared image set such that the user can be aware of the bleeding status of the target object on the infrared image set and decide on an appropriate procedure for coping with the internal bleeding. Besides, the method as shown in FIG. 10A can contribute to the technical field about processing and analyzing the surgical image set.

Referring to FIG. 10B, FIG. 10B is a schematic diagram illustrating one of the surgical image set 510 with the surgical condition information and one of the infrared image set 610 with the surgical condition information according to an embodiment of the present disclosure. FIG. 10B is a colored figure.

In some embodiments, the surgical condition information (such as the bleeding point identification 530) that is generated by the computing device as shown in FIG. 2 may be displayed with the surgical image set 510 synchronously in a displaying region 500. In some embodiments, the surgical condition information (such as the bleeding point identification 530) that is generated by the computing device as shown in FIG. 2 may be displayed with the infrared image set 610 synchronously in a displaying region 600.

Based on the above, the present disclosure can clearly display the surgical condition information (such as the bleeding point identification 530) with the surgical image set 510 and/or the infrared image set 610 such that the user can be immediately aware of the bleeding status of the target object and decide on an appropriate procedure for coping with the internal bleeding.

In some embodiments, the steps of the method of generating surgical condition information described above may be designed as a computer program product. After the computing device executes the computer program product, the computing device can implement the method of generating surgical condition information described above.

In some embodiments, the computer program product may be stored in a non-transitory computer-readable storage medium in a series of particular codes or a series of particular instruction sets. After the computing device loads and executes the computer program product that is stored in the non-transitory computer-readable storage medium by the computing device, the computing device can implement the method of generating surgical condition information described above. In some embodiments, the non-transitory computer-readable storage medium may be, for example, a hard disk, a CD-ROM, a magnetic disk, or a USB disk, but is not limited thereto.

While the present disclosure has been described by means of specific embodiments, numerous modifications and variations could be made thereto by those skilled in the art without departing from the scope and spirit of the present disclosure set forth in the claims. Therefore, the protection of the present application shall be as defined in the claims instead of the contents disclosed in the specification.

What is claimed is:

1. A method of generating surgical condition information by a computing device based on a surgical image set associated with a surgical site, the method comprising:
receiving the surgical image set; and
automatically generating the surgical condition information based on the surgical image set,
wherein the surgical condition information contains a bleeding warning, a bleeding point identification, a blood flow path indication, a prompting bleeding point, a bleeding amount indication, or a bleeding speed indication.

2. The method according to claim 1, wherein the automatically generating the surgical condition information based on the surgical image set comprises:
inputting the surgical image set into a first image identifying model; and
outputting the bleeding warning by the first image identifying model,
wherein the bleeding warning is automatically generated based on the surgical image set by the first image identifying model, and
wherein the first image identifying model is a deep learning model that has been trained by plural pieces of first training images.

3. The method according to claim 1, wherein the automatically generating the surgical condition information based on the surgical image set comprises:
inputting the surgical image set into a second image identifying model; and
outputting the bleeding point identification by the second image identifying model,
wherein the bleeding point identification is automatically generated based on the surgical image set by the second image identifying model, and
wherein the second image identifying model is a deep learning model that has been trained by plural pieces of second training images.

4. The method according to claim 3, further comprising:
marking the bleeding point identification that is corresponding to the surgical image set on the surgical image set.

5. The method according to claim 1, wherein the automatically generating the surgical condition information based on the surgical image set comprises:
inputting a first image and a second image into an optical flow tracking calculating model; and
outputting the blood flow path indication by the optical flow tracking calculating model,
wherein the surgical image set contains the first image and the second image, and
wherein the blood flow path indication is automatically generated based on the first image and the second image by the optical flow tracking calculating model.

6. The method according to claim 5, further comprising:
generating the prompting bleeding point based on the blood flow path indication.

7. The method according to claim 5, further comprising:
marking the blood flow path indication on the surgical image set.

8. The method according to claim 1, wherein the automatically generating the surgical condition information based on the surgical image set comprises:
inputting the surgical image set into a third image identifying model;
outputting a bleeding area indication by the third image identifying model; and
converting the bleeding area indication into the bleeding amount indication,
wherein the bleeding area indication is automatically generated based on the surgical image set by the third image identifying model, and
wherein the third image identifying model is a deep learning model that has been trained by plural pieces of third training images.

9. The method according to claim 7, further comprising:
generating the bleeding speed indication based on the bleeding amount indication that is corresponding to a third image and the bleeding amount indication that is corresponding to a fourth image,
wherein the surgical image set contains the third image and the fourth image.

10. The method according to claim 1, further comprising:
receiving an infrared image set;
performing an image processing for the infrared image set; and
displaying the surgical condition information on the infrared image set.

11. A computing device of generating surgical condition information, wherein a surgical condition information is generated based on a surgical image set that is associated with a surgical site by the computing device, wherein the computing device signally connecting with an endoscope device via a signal transmitting path, and wherein the endoscope device being configured to provide a surgical image set, the computing device comprising:
a processing module; and
a storage module, configured to signally connect with the processing module;
wherein a code is stored in the storage module, and after the processing module executes the code stored in the storage module, the computing device performs the steps as described below:
receiving the surgical image set; and
automatically generating the surgical condition information based on the surgical image set,
wherein the surgical condition information contains a bleeding warning, a bleeding point identification, a blood flow path indication, a prompting bleeding point, a bleeding amount indication, or a bleeding speed indication.

12. The computing device according to claim 11, wherein the automatically generating the surgical condition information based on the surgical image set comprises:
inputting the surgical image set into a first image identifying model; and
outputting the bleeding warning by the first image identifying model,
wherein the bleeding warning is automatically generated based on the surgical image set by the first image identifying model, and
wherein the first image identifying model is a deep learning model that has been trained by plural pieces of first training images.

13. The computing device according to claim 11, wherein the automatically generating the surgical condition information based on the surgical image set comprises:
inputting the surgical image set into a second image identifying model; and
outputting the bleeding point identification by the second image identifying model,
wherein the bleeding point identification is automatically generated based on the surgical image set by the second image identifying model, and
wherein the second image identifying model is a deep learning model that has been trained by plural pieces of second training images.

14. The computing device according to claim 13, wherein the computing device further performs the step as described below:
marking the bleeding point identification that is corresponding to the surgical image set on the surgical image set.

15. The computing device according to claim 11, wherein the automatically generating the surgical condition information based on the surgical image set comprises:
inputting a first image and a second image into an optical flow tracking calculating model; and
outputting the blood flow path indication by the optical flow tracking calculating model,
wherein the surgical image set contains the first image and the second image, and
wherein the blood flow path indication is automatically generated based on the first image and the second image by the optical flow tracking calculating model.

16. The computing device according to claim 15, wherein the computing device further performs the step as described below:
generating the prompting bleeding point based on the blood flow path indication.

17. The computing device according to claim 15, wherein the computing device further performs the step as described below:
marking the blood flow path indication on the surgical image set.

18. The computing device according to claim 11, wherein the automatically generating the surgical condition information based on the surgical image set comprises:
inputting the surgical image set into a third image identifying model;
outputting a bleeding area indication by the third image identifying model; and
converting the bleeding area indication into the bleeding amount indication,
wherein the bleeding area indication is automatically generated based on the surgical image set by the third image identifying model, and
wherein the third image identifying model is a deep learning model that has been trained by plural pieces of third training images.

19. The computing device according to claim 17, wherein the computing device further performs the step as described below:
generating the bleeding speed indication based on the bleeding amount indication that is corresponding to a third image and the bleeding amount indication that is corresponding to a fourth image,
wherein the surgical image set contains the third image and the fourth image.

20. The computing device according to claim 11, wherein the computing device further performs the steps as described below:
receiving an infrared image set;
performing an image processing for the infrared image set; and
displaying the surgical condition information on the infrared image set.
